(19) United States
(12) Patent Application Publication
(10) Pub. No.: US 2021/0038483 A1
(43) Pub. Date: Feb. 11, 2021

(54) FILM-FORMING SYSTEM WITH BARRIER EFFECT, IN PARTICULAR AGAINST AIR POLLUTION, OF NATURAL ORIGIN AND FOR USE IN COSMETICS

(71) Applicant: ROQUETTE FRERES, LESTREM (FR)

(72) Inventors: Léon MENTINK, LILLE (FR); Daniel WILS, MORBECQUE (FR); Sophie PIOT, PARIS (FR)

(21) Appl. No.: 17/048,283

(22) PCT Filed: Apr. 17, 2019
(86) PCT No.: PCT/FR2019/050920

§ 371 Date: Oct. 16, 2020

(30) Foreign Application Priority Data
Apr. 17, 2018 (FR) 1853350

(51) Int. Cl. A61K 8/02; A61K 8/73; A61Q 17/00; A61Q 19/08

(52) U.S. Cl. CPC A61K 8/0204; A61K 8/731; A61K 8/732; A61K 2800/10; A61Q 17/00; A61Q 19/08; A61K 2800/594; A61K 8/737

(57) ABSTRACT

The subject matter of the application is a film-forming system, preferably for cosmetic use, made up of at least one pregelatinsed starch and of at least one non-starch polysaccharide selected from: gums of plant origin, preferably gums originating from algae or plants, gums of microbial origin, and cellulose derivatives. The film-forming system according to the invention provides a barrier effect, in particular against air pollution. The application also relates to a method for manufacturing such a film-forming system and to the cosmetic compositions, in particular topical compositions, containing said film-forming system, such as skincare products, hair care products, make-up products, solar protection products, hygiene products or perfumes.

The present patent application relates to the use in topical application of a film-forming system of natural origin, which can be used in particular as an agent for combating atmospheric pollution, having the effect of creating a film at the surface of the skin or of the hair acting as a barrier to particles of polluting material.

The patent application also relates to a cosmetic composition having a barrier and pollution-combating effect comprising the film-forming system in a physiologically acceptable medium. The present patent application provides a solution to two current cosmetic needs: to provide a product which is as natural as possible, and to protect the skin from environmental attacks, in particular atmospheric pollution.

Cosmetic products are formulated products produced from the mixing, combining or shaping of multiple ingredients. Today, for reasons of public health, protection of the environment and sustainability, it is increasingly sought to formulate them from natural ingredients instead of synthetic ones. Natural polymers, in the form of gums and resins, which have been used for a long time as water-soluble binders, film-forming agents and also thickeners, are for this reason enjoying renewed interest.

Environmental pollution, and more particularly that atmospheric pollution known under the name of “smog”, is composed of inorganic particles and fibers which can comprise heavy metals, covered and linked together by toxic or carcinogenic organic compounds, such as polycyclic aromatic hydrocarbon compounds, furans or aldehydes, which can even be combined with pathogenic microorganisms. Such particles have sizes ranging from less than 1 μm up to 500 μm. The smaller these particles, the greater their toxicity. According to a study carried out by L'Oreal, particles ranging in size from 2.5 μm to 10 μm are the most harmful: they penetrate deep into the epidermis, where they cause serious chemical damage.

Atmospheric pollution causes premature aging of the skin. It has been demonstrated that the aging process caused by this pollution is directly linked to the peroxidation of epidermal lipids, to oxidative stress, to apoptosis and to cell damage caused by UVB radiation, bringing about the appearance of blackheads, a decrease in the radiance of the skin and an increase in the sensitivity of the skin.

Protecting the skin from environmental pollution is thus a new cosmetic target for protecting the appearance and the health of the skin, while maintaining a pleasant texture from a sensory point of view on the skin, the nails or the hair.

Initially, the industry resorted to polymers of natural origin for this film-forming function having a pollution-combating barrier effect. However, the latter exhibited a certain number of disadvantages, in particular in terms of color, odor, purity, consistency of efficiency and viscosity stability. These reasons led to them being replaced by synthetic or semisynthetic polymers.

In this respect, carbomers, which are widely used in cosmetics, are known. The Carbopol® range developed by LUBRIZOL is an example thereof. Mention may in particular be made of the product Carbopol® Ultrez 10NF, which is a copolymer of polyethylene glycol and of an acid ester with a long alkyl chain, created to provide film-forming properties to a wide variety of cosmetic formulas.

Nevertheless, the cosmetics industry must nowadays face up to new challenges in terms of protecting the environment, of preserving our fossil resources, of carbon footprint and of safety and protection of consumers. From this point of view, it is reconsidering its formulations and returning as much as possible to the use of solutions of natural origin for the formulation of its products. Science and technology are progressing in this field for the purpose of proposing reliable and successful technical solutions, enabling the formulator to achieve effective film-forming functions in the products which he makes, with natural ingredients, which are very environmentally friendly and consumer friendly.

In this regard, the applicant company has succeeded in developing a new film-forming system having a barrier effect, in particular against atmospheric pollution, starting from a mixture of at least one pregelatinized starch, and of at least one non-starchy polysaccharide chosen from gums of vegetable origin, gums of microbial origin, and cellulose derivatives. Starting with this combination, the applicant company has succeeded in developing a film-forming system which is particularly well-suited to cosmetic formulations.

The film-forming system according to the invention is capable of being applied to the human epidermis or any other external part of the human body, such as in particular the hair, in the form of an aqueous composition which, after drying, results in the formation of a film at the surface of the epidermis. This film is a protective barrier, forming a double skin, which prevents or reduces contact of the epidermis or the body hair system with atmospheric pollution particles. When the epidermis or the body hair system, thus covered by the film, is exposed to microparticles, rinsing with water makes it possible to remove a portion of the microparticles deposited on the film, whereas, in the absence of film, no removal by washing with water is possible. The film-forming system of the invention provides a film having protective properties for the skin and the body hair system with respect to polluting atmospheric particles, in particular to microparticles: reduction of exposure to these particles, and reduction in the adhesion of these particles to the epidermis or the body hair system. Furthermore, compared to the films formed with a film-forming agent of petrochemical origin, such as polyacrylates, the reduction in the adhesion is greater with the film-forming system according to the invention.

The film-forming system according to the invention also makes it possible to confer a film-forming function on aqueous cosmetic compositions, in particular aqueous solutions, and to stabilize them, this being achieved in a completely unexpected and synergistic manner between its various constituents. In this respect, the viscosity obtained in the presence of the mixture is much higher than that observed for each of the constituents taken separately, or for the combinations of only two of these constituents.

The film-forming system according to the invention advantageously exhibits a viscosity which can be adjusted and which is astutely chosen to allow its use in a sufficient concentration in the cosmetic composition without leading to an excessively thickened texture of the latter and to make it possible to obtain, after application, the best film-forming effect and a film which is the most continuous possible on the skin or the hair.

Furthermore, the film-forming system according to the invention makes it possible to obtain a very great stability of the viscosity over time, without notable change in texture, this being the case over durations of several months. Such a result is particularly advantageous, and makes it possible to achieve the high level of stability required for cosmetic products in which said film-forming system is liable to be used.

From the sensory point of view, the film-forming system according to the invention also results in particularly advantageous properties by making it possible to obtain cosmetic products with a creamy texture which, after application, give a particularly soft and pleasant feel.

For this reason, the film-forming system according to the invention makes it possible to obtain both a barrier and protective film against pollution but also to stabilize gels or emulsions, such as care or makeup creams, to adjust their textures and to improve their sensory properties by advantageously conferring on them a nongreasy, nonstringy and particularly soft and fresh feel.

Consequently, a first subject matter of the present invention consists of a film-forming system, preferentially for cosmetic use, constituted:
a) of at least one pregelatinized starch,
b) of at least one non-starchy polysaccharide chosen from:
   i. gums of vegetable origin, preferentially gums resulting from algae or from plants,
   ii. gums of microbial origin,
   iii. cellulose derivatives.

According to a first embodiment, the film-forming system of the present invention can take the form of a system constituted:
of at least one pregelatinized starch,
and of at least one non-starchy polysaccharide chosen from gums of vegetable origin and gums of microbial origin.

According to a second embodiment, the film-forming system of the present invention can take the form of a system constituted:
of at least one pregelatinized starch,
of at least one non-starchy polysaccharide chosen from gums of vegetable origin and gums of microbial origin,
and of at least one non-starchy polysaccharide chosen from cellulose derivatives.

A second subject matter of the present invention relates to a process for the preparation of a film-forming system, through the stages of:
a) providing an aqueous solution,
b) heating the aqueous solution to a temperature of between 20° C. and 80° C., preferentially between 20° C. and 50° C. and more preferentially between 20° C. and 30° C.,
c) introducing, with stirring, into the aqueous solution, at least one pregelatinized starch and at least one non-starchy polysaccharide chosen from gums of vegetable origin, gums of microbial origin or cellulose derivatives, in order to obtain a medium.

A third subject matter of the present invention relates to a cosmetic composition comprising the film-forming system according to the invention.

A fourth and final subject matter of the present invention relates to the cosmetic use of a film-forming system according to the invention. More particularly, the present invention relates to the use of the film-forming system according to the invention in a topical composition, preferentially for use on the human epidermis, to provide said topical composition with a barrier property, preferentially one combating atmospheric pollution.

The film-forming system according to the present invention is an at least binary mixture comprising at least 50%, or better still 60%, by weight, with respect to the total weight, at least one pregelatinized starch. Such starches can comprise amylose and amylopectin in variable proportions. Particularly preferred are the pregelatinized starches derived from corn, potato, wheat, rice, peas, oats, lentils, faba beans, broad beans, beans, chickpeas, or combinations thereof. Preferably, this starch is a waxy starch, that is to say a starch rich in amylopectin and poor in amylose, preferentially containing at least 95 m % of amylopectin. It can in particular be a waxy corn, potato or rice starch.

Pregelatinized starches are generally prepared by thermal, chemical or mechanical techniques liable to bring about simple swelling, partial splitting, indeed even complete dissolution, of the starch granules so that they become partially or completely soluble in water according to a “cold” process, that is to say by dispersion in water at a water temperature of less than 45° C., better still of less than 35° C. and even better still in the region of ambient temperature. Thus, preferably, the pregelatinized starch no longer exhibits or virtually no longer exhibits granules displaying a Maltese cross in polarized light.

The preferred techniques for obtaining a pregelatinized starch are techniques of cooking/drying suspensions of starch in an aqueous medium, such as in particular atomization, cooking on a drum or extrusion. Autoclaving or indirect heating on a heat exchanger are cooking processes which are also possible and tend to produce complex colloidal dispersions consisting of intact, fragmented and swollen granules. Examples of processes for the preparation of such starches will be found in the documents U.S. Pat. Nos. 3,086,890, 3,607,394 or FR 2 822 471.

This pregelatinized starch may or may not be modified before or after pregelatinization, that is to say after application of the cooking/drying treatment described above.

When it is desired to retain an unmodified pregelatinized starch, that is to say a starch not grafted chemically or enzymatically, this pregelatinized starch will advantageously be chosen from waxy starches, that is to say starches rich in amylopectin and poor in amylose. It can in particular be a waxy corn, potato or rice starch, the viscosity of which may have been adjusted by simple hydrolysis according to techniques well known to a person skilled in the art. When it is desired to have available a starch having very highly film-forming properties while being very stable in the formulation of cosmetic products, it will be possible to then preferably choose a modified pregelatinized starch. In terms of modification, it may then be a matter of one or more modification(s) by a physical route, physicochemical route, chemical route or enzymatic route. It may in particular be a treatment, or several treatments combined with one another, of dextrinization, of hydrolysis by the acid route, oxidizing route or enzymatic route, of carboxymethylation, of hydroxypropylation, of hydroxyethylation, of acetylation, of octenylsuccinylation, of cationization, of crosslinking or of grafting. Preferably, the pregelatinized starch is chosen from modified starches, in particular dextrinized, hydrolyzed, carboxymethylated, hydroxypropylated, acetylated, octenylsuccinated or cationic, pregelatinized starches. More preferentially, the pregelatinized starch is chosen from carboxymethylated, hydroxypropylated, acetylated, octenylsuccinated pregelatinized starches.

It should be noted, however, that the thermal or mechanical techniques liable to give rise to simple swelling, partial splitting, indeed even complete dissolution, of the starch granules so that they become soluble in water according to a “cold” process are not necessary when certain physicochemical or chemical modification treatments applied to the starch are sufficiently intensive. This is because it is possible to use, as pregelatinized starch within the meaning of the invention, products which have solely been dextrinized, hydrolyzed, cationized, hydroxypropylated or carboxymethylated no longer exhibiting or virtually no longer exhibiting granules displaying a Maltese cross in polarized light as a simple result of their modification.

In particular, it is preferred to choose, from pregelatinized starches, nonionic pregelatinized starches and especially those of the range sold by the applicant company under the brand name PREGEFLO®, GLUCIDEX®, STABILYS® or TACKIDEX®. Examples of such starches which are the most preferred are acetylated or hydroxypropylated starches, such as, for example, the acetylated starch PREGEFLO® CH 40.

The film-forming system according to the invention is constituted of a second component which is a non-starchy polysaccharide. This component can be chosen from: gums of vegetable origin, preferentially gums resulting from algae or from plants; gums of microbial origin; or cellulose derivatives. The term “non-starchy polysaccharide” is understood, by the applicant company, to mean polysaccharides of cellulose origin or of microbial origin, such as exopolysaccharides, which are predominantly, indeed even completely, constituted of saccharides bonded together via glycoside bonds in the β conformation and which can exhibit a linear or branched structure.

Mention may in particular be made, as gum of vegetable origin, of:
gums resulting from plant seeds or exudates, such as gum arabic, konjac gum, guar gum, locust bean gum, tragacanth gum, tara gum, cassia gum, karaya gum, psyllium gum, pectin and pectates, or their derivatives and mixtures;
gums extracted from algae, such as agar, galactomannans, alginates or carrageenans, or their derivatives and mixtures.

Mention may in particular be made, as gum of microbial origin, of gums resulting from a microbial fermentation, such as xanthans, gellans, mannans, scleroglucans or their derivatives and mixtures, and preferably a xanthan gum.

Preferably, this gum of vegetable or microbial origin employed in the film-forming system according to the invention is a nonionic polysaccharide. Preference is given to gums resulting from fermentation, such as xanthans, gellans, mannans and scleroglucans, and in particular xanthans and scleroglucans, and more particularly xanthans. Such xanthan gums generally have a molecular weight of between 1 000 000 and 50 000 000 Da. Mention may be made, among the possible commercial products, for example, of the product Xanthan Gum FNCS-PC from Jungbunzlauer International AG, the product Keltrol® CG-T from CP Kelco, the product Cosphaderm® X 17 from Cosphatec, the product Kahlgum 6673 FEE—Xanthan Gum from KahlWax, the products Rhodicare® S and Rhodicare® XC from Solvay and the product VANZAN® NF-C from Vanderbilt Minerals.

Cellulose derivatives which may be accepted are modified celluloses, in particular methylcelluloses, hydroxyalkylcelluloses, ethylhydroxyethylcelluloses, methylethylcelluloses, carboxymethylcelluloses, hydroxypropylcelluloses or hydroxypropylmethylcelluloses, carboxymethylcelluloses and hydroxyethylcelluloses being preferred, hydroxyethylcelluloses being the most preferred. Mention may in particular be made of the following commercial products: Natrosol™ 250 HHR PC from Ashland Specialty Chemical; Espesante CH from Chemir; Tylose® H 15 YG4 from SE Tylose and Cellosize™ HEC QP 40 from DowDuPont (Dow).

Although the relative proportions between the different constituents of the binary mixture are not fundamental, it is preferable, in order for the film-forming system to be as effective as possible, for the pregelatinized starch to be predominant with respect to the whole of the film-forming system, i.e. thus to represent more than 50% by weight and preferably more than 60% by weight, indeed even more than 70% by weight, of the whole of the film-forming system.

Thus, according to a first embodiment, the film-forming system according to the invention comprises, and preferably is constituted of:
1 to 12 parts by dry weight of at least one pregelatinized starch,
0.01 to 5 parts, preferably 0.01 to 2 parts, by dry weight of at least one non-starchy polysaccharide chosen from gums of vegetable origin, gums of microbial origin and cellulose derivatives.

According to a second embodiment, the film-forming system according to the invention comprises, and preferably is constituted of:
a) 1 to 12 parts by dry weight of at least one pregelatinized starch,
b) 0.01 to 2 parts of at least one gum of vegetable or microbial origin.

More preferentially, it comprises, or better still is constituted of:
a) 5 to 11 parts by dry weight of at least one pregelatinized starch,
b) 0.1 to 1 part of at least one gum of vegetable or microbial origin.

Very preferentially, it comprises, or better still is constituted of:
a) 6 to 10 parts by dry weight of at least one pregelatinized starch,
b) 0.2 to 0.8 parts of at least one gum of vegetable or microbial origin.

And, according to a third embodiment, the film-forming system according to the invention comprises, and preferably is constituted of:
a) 1 to 12 parts by dry weight of at least one pregelatinized starch,
b) 0.01 to 2 parts of at least one gum of vegetable or microbial origin,
c) and 0.01 to 3 parts of at least one cellulose derivative.

More preferentially, it comprises, or better still is constituted of:
a) 5 to 11 parts by dry weight of at least one pregelatinized starch,
b) 0.1 to 1 part of at least one gum of vegetable or microbial origin,
c) 0.1 to 2 parts of at least one cellulose derivative.

Very preferentially, it comprises, or better still is constituted of:
a) 6 to 10 parts by dry weight of at least one pregelatinized starch,
b) 0.2 to 0.8 parts of at least one gum of vegetable or microbial origin,
c) 1 to 1.5 parts of at least one cellulose derivative.

According to an advantageous alternative form, the film-forming system according to the invention exhibits, in water, a Brookfield viscosity, measured at 20° C., of between 1000 and 20 000 mPa·s, preferably of between 1500 and 15 000 mPa·s and very preferentially of between 2000 and 10 000 mPa·s, for a concentration ranging from 1% to 10%, preferably ranging from 2% to 6%. Typically, this viscosity will be between 1000 and 15 000 mPa·s, better still between 2000 and 10 000 mPa·s, for a concentration of 4% in water. The Brookfield viscosity is measured with a Brookfield DV-II+Pro viscometer, at a speed of 20 rpm, with a spindle suited to the viscosity measured: the spindle chosen is the SP3 spindle when the viscosity is less than or equal to 5000 mPa·s, the SP4 spindle when the viscosity is between 5000 mPa·s and 7000 mPa·s and the SP5 spindle when the viscosity is greater than or equal to 7000 mPa·s.

The film-forming system according to the invention makes it possible to reduce the exposure of the epidermis and of the hair to polluting atmospheric particles, by preventing or reducing the adhesion of the microparticles. This is because, when it is applied to the skin by means of an aqueous cosmetic composition, the film-forming system results, after evaporation of the water, in the formation of a film on the surface of the epidermis. This film acts as a physical barrier between the part of the body and the atmosphere which surrounds it, with the result that a portion of the atmospheric particles to which it is exposed, in particular those of micrometric size, known as microparticles, in particular with a size of between 1 μm and 10 μm, cannot pass through it, and thus cannot come into contact with the underlying parts, such as in particular the epidermis. This reduces the number of pollution particles liable to be deposited, in particular on the virgin epidermis, to penetrate therein and to cause damage thereto: the film-forming system according to the invention thus makes it possible to limit the harmful effects of atmospheric pollution on the epidermis or any other part of the body. A second advantage of the film-forming system according to the invention is to facilitate the removal of the microparticles deposited, for example on the epidermis, by washing, in particular by rinsing with water. After application to and drying on the epidermis, the barrier film obtained also exhibits the distinguishing feature of not retaining the particles. Rinsing of the film with water is sufficient to remove a portion of the particles deposited on the film. Compared to petrochemical film-forming agents, the film-forming system according to the invention exhibits an increased effectiveness in reducing the adhesion of the particles to the skin.

Another subject matter of the present invention consists of a process for the manufacture of a film-forming and stable system, through the stages of:
a) providing an aqueous solution,
b) heating the aqueous solution to a temperature of between 20° C. and 80° C., preferentially between 20° C. and 50° C. and more preferentially still to a temperature close to ambient temperature,
c) introducing, with stirring, into the aqueous solution, at least one pregelatinized starch and at least one non-starchy polysaccharide chosen from gums of vegetable origin, gums of microbial origin or cellulose derivatives, so as to obtain a medium.

A person skilled in the art will know how to adjust the stirring speed of the medium, in particular as a function of the amount of the ingredients to be dispersed. However, a stirring speed of between 1000 and 5000 revolutions per minute appears to be entirely acceptable. Furthermore, it is of course understood that the process according to the invention incorporates all the characteristics listed above relating to the binary system manufactured according to said process.

The film-forming system according to the invention proves to be very stable and nonallergenic to the skin. It furthermore offers the advantage of exhibiting of giving consistency of viscosity and of texture, independently of the pH or of the presence of electrolytes. In other words, this system is not greatly affected by the pH of the medium, or by the presence of monovalent, divalent or trivalent salts. This criterion is all the more important since, in general, products for cosmetic use and in particular for topical application are liable to be subjected or exposed to pH variations. Thus, the pH of the skin, which is slightly acidic, for example varies between 4 and 6. Having available a product which does not exhibit a particular limit for use in terms of pH or of presence of salts thus represents a very great technical advantage for a cosmetic composition.

Lastly, a final subject matter according to the invention consists of a cosmetic composition containing the film-forming and in particular stable system according to the invention. This is because the film-forming and in particular stable system in accordance with the invention makes possible the easy production of emulsions which are simultaneously very stable and very fine, with adjustable textures and exhibiting a fresh, silky and nongreasy feel, even for high contents of dispersed fatty phase. Preferably, the cosmetic composition comprises the film-forming system in a concentration of between 1% and 10% by weight, more preferentially of between 1.5% and 8% by weight and better still between 2% and 6% by weight.

Said cosmetic composition can in particular be a care product for the skin, such as a moisturizing, antiwrinkle, antiaging, slimming or firming composition, a body balm or a beauty mask and be provided in the form of thickened solutions, of gels, of milks, of creams, of suspensions, of aerosols or of foams.

Said cosmetic composition can in particular be a makeup product for the eyes, such as a mascara or a liner, or a makeup product for the face, such as a powder or a foundation, for the face, or a makeup product for the nails, such as a varnish, or a makeup product for the lips, such as a lipstick or a lip gloss.

Said cosmetic composition can in particular be a sun product, such as a protective product or a self-tanning product.

Said cosmetic composition can in particular be a body hygiene product, such as a soap, a hair remover or a deodorant.

Said cosmetic composition can in particular be a hair product, such as a shampoo, a coloring, a dye, a permanent-waving product, a lotion for combating hair loss, a lacquer or a fixative.

Said cosmetic composition can in particular be a fragrance, an eau de toilette or an eau de parfum.

The examples which follow will make possible a better understanding of the present invention, without, however, limiting the scope thereof.

### EXAMPLES

Throughout the examples, different formulations have been produced, with the following products:
Pregeflo® CH 40, sold by Roquette Freres
Pregeflo® CR 3510, sold by Roquette Freres: it is a pregelatinized and hydroxypropylated waxy corn starch having a hydroxypropyl content of approximately 7 m %,
Xanthan gum, sold by CP Kelco under the name Keltrol® CG-T
Hydroxyethylcellulose (HEC), sold by Ashland Specialty Chemical under the name Natrosol™ 250 HHR PC

All the viscosities are determined from a Brookfield DV-II+Pro viscometer, at a speed of 20 rpm, with a spindle suited to the viscosity measured: the spindle chosen is the SP3 spindle when the viscosity is less than or equal to 5000 mPa·s, the SP4 spindle when the viscosity is between 5000 mPa·s and 7000 mPa·s and the SP5 spindle when the viscosity is greater than or equal to 7000 mPa·s.

### Example 1: Preparation of the O/W Emulsions

This example illustrates the preparation of emulsions of oil-in-water type containing, as film-forming agent, either a film-forming agent of petrochemical origin (table 1) or a film-forming agent according to the invention (tables 2 and 2a).

TABLE 1
emulsion A, reference, with film-forming
agent of petrochemical origin
Oily phase

| | |
|---|---|
| Helianthus annuus seed oil | 15% |

Aqueous phase

| | |
|---|---|
| Water | q.s. for 100% |
| Film-forming agent: Carbopol | 0.3% |
| Ultrez 10NF - Lubrizol | |
| Preservative: Sepicide HB - Seppic | 1% |
| Emulsifier: Montanov L - Seppic | 3% |

TABLE 2
emulsion INV1 with film-forming
system according to the invention
Oily phase

| | |
|---|---|
| Helianthus annuus seed oil | 15% |

Aqueous phase

| | |
|---|---|
| Water | q.s. for 100% |
| Film-forming system: | |
| Pregeflo CH 40 - Roquette Frères | 3.3% |
| Keltrol ® CG-T - CP Kelco | 0.2% |
| Natrosol ™ 250 HHR PC - Ashland | 0.5% |
| Specialty Chemical | |
| Preservative: Sepicide HB - Seppic | 1% |
| Emulsifier: Montanov L - Seppic | 3% |

TABLE 2a
emulsion INV2 with film-forming
system according to the invention
Oily phase

| | |
|---|---|
| Helianthus annuus seed oil | 15% |

Aqueous phase

| | |
|---|---|
| Water | q.s. for 100% |
| Film-forming system: | |
| Pregeflo ® CR 3510 - Roquette Frères | 3.8% |
| Keltrol ® CG-T - CP Kelco | 0.2% |
| Preservative: Sepicide HB - Seppic | 1% |
| Emulsifier: Montanov L - Seppic | 3% |

The procedure for the preparation of an emulsion is as follows: the aqueous phase is prepared by dispersing the film-forming agent in water at 35-40° C. with stirring with a deflocculating paddle at 1000 revolutions per minute for at least 10 minutes. The emulsifier is subsequently added with stirring at 35-40° C. Separately, the oily phase is heated to 35-40° C. The oily phase is subsequently emulsified in the aqueous phase at 35-40° C. with stirring with a deflocculating paddle at 1500 revolutions per minute for 15 minutes. Finally, the preservative is added. The emulsion is kept stirred until it is at ambient temperature.

The emulsions prepared according to this procedure and the compositions given in tables 1, 2 and 2a form creams capable of being applied to the skin. After having been applied, they form a protective film, the barrier properties of which to atmospheric particles are evaluated in the following example.

TABLE 3
characteristics of the emulsions prepared

| Emulsion | Viscosity (mPa · s) | Particle size (μm) | pH |
|---|---|---|---|
| A | 10 000 | 10 to 20 | 6 |
| INV1 | 13 000 | 10 | 6 |

### Example 2: Pollution-Combating Barrier Effect

This example compares the performance qualities of the barrier effects in combating pollution by microparticles for the three emulsions prepared in example 1.

To do this, tests were carried out on human epidermides of 10 volunteers aged from 18 to 65 years, using as experimental model charcoal particles with sizes of between 1 μm and 5 μm, described as microparticles. These charcoal microparticles suitably model actual polluting microparticles, such as internal combustion engine exhaust gas particles.

### Preparation of the Charcoal Microparticles:

Prior to the tests, a sufficient amount of charcoal microparticles was prepared by subjecting charcoal to the action of a domestic grinder for 10 minutes. This grinding provides particles of micrometric size having a size distribution distributed predominantly between 1 μm and 5 μm.

### Procedure of the Test on the Epidermis of the Volunteers:

Three zones of 1 cm by 1 cm were delimited on the forearm of each volunteer. For each zone thus delimited, the following two measurements are carried out: a measurement of counting the number of black microparticles by means of a photograph taken with a Dino-Lite digital microscope, then processed by image analysis, as well as a measurement of the colorimetry with a Minolta© CR-200 chromameter.

### Counting of the Microparticles:

The high resolution photographs taken with the Dino Lite digital microscope are processed with the GIMP (GNU Image Manipulation Software) image processing software. First, a projection along an axis (black and white) was carried out in order to bring out the black microparticles and to standardize the images. The number of black pixels was subsequently counted with the GIMP software.

### Measurement of the Colorimetry:

The Minolta CR-200 chromameter is a device for the unbiased measurement of the color of surfaces. It provides a result composed of three coordinates L*, a* and b* in the CIE 1976 color space (also called the CIELAB color space). Only the parameter L*, which characterizes the lightness of the color, was made use of: L*=0 corresponds to the color black, L*=100 indicates a white color.

### Carrying Out the Test on the Volunteers:

The measurements of counting microparticles and of colorimetry are carried out before the application of the emulsions, that is to say on virgin skin (T0 measurements).

Three zones were covered with the emulsions to be tested, one with the emulsion A, one with the emulsion INV1 and one with the emulsion INV2. For this, approximately 2 mg of emulsion per cm² of skin were applied by depositing the required amount with a pipette and by then spreading with a spatula. The volunteers subsequently waited 20 minutes to allow the emulsion to dry. The third zone is kept blank in order to constitute the control zone. The measurements were carried out (T1 measurements).

The charcoal particles were applied to the 3 zones by dabbing them with a makeup sponge impregnated with particles and then the measurements were carried out (T2 measurements). The 3 zones were subsequently rinsed by causing 100 ml of water to stream over the entire surface of each zone and then the measurements were carried out (T3 measurements). The results presented in the following table were obtained:

TABLE 4
mean results of the number of black pixels obtained
with regard to the panel of volunteers

| Moment | Mean number of black pixels | | | |
|---|---|---|---|---|
| of the | | Emulsion | Emulsion | Emulsion |
| Measurements | Control | A | INV1 | INV2 |
| T0 | 213 320 | 218 297 | 226 996 | 237 653 |
| T1 | 229 085 | 223 261 | 280 552 | 295 122 |
| T2 | 594 398 | 554 486 | 657 915 | 671 473 |
| T3 | 591 123 | 525 848 | 633 858 | 519 031 |

TABLE 4a
mean results of the parameter L* obtained
with regard to the panel of volunteers

| Moment | Mean parameter L* | | | |
|---|---|---|---|---|
| of the | | Emulsion | Emulsion | Emulsion |
| Measurements | Control | A | INV1 | INV2 |
| T0 | 62.87 | 63.06 | 64.05 | 62.14 |
| T1 | 63.60 | 63.25 | 63.61 | 62.55 |
| T2 | 20.01 | 21.45 | 22.44 | 20.77 |
| T3 | 24.81 | 33.46 | 36.42 | 37.93 |

The pollution-combating barrier effect was then evaluated by calculating the percentage of variation in the number of black pixels and in the lightness between T3 and T2, with respect to the values at T2.

TABLE 5
mean percentages of variation between T3 and T2, with respect to T2
Mean number of black pixels

| | Emulsion | Emulsion | Emulsion |
|---|---|---|---|
| Control | A | INV1 | INV2 |
| −0.78% | −5.97% | −8.64% | −22.7% |

TABLE 5a
mean percentages of variation between T3 and T2, with respect to T2
Mean parameter L*

| | Emulsion | Emulsion | Emulsion |
|---|---|---|---|
| Control | A | INV1 | INV2 |
| 24.81% | 59.95% | 64.17% | 82.6% |

The absence of barrier effect was confirmed on the control zone: rinsing with water does not remove the deposited microparticles (variation of less than 1% not significant). The rinsing made it possible to increase the lightness by 24.81%, which is significant, probably as a result of the removal of microparticles not detected during the counting measurement, due to a size below the detection limit of the digital microscope.

As expected, the barrier effect is clearly demonstrated on the zone covered with the emulsion A, containing the petrochemical film-forming agent: the number of black pixels has decreased by 5.97%, which is indeed significant, and the lightness has increased by 59.95%, i.e. 35% more than the control zone.

Surprisingly and unexpectedly, the barrier effect was also observed on the zone covered with the emulsion INV1, containing the film-forming system according to the invention, and is even improved with respect to the zone covered with the emulsion A: the reduction in the number of black pixels reaches 8.64%, i.e. 2.7% more than the emulsion A; the lightness reaches 64.17%, i.e. 4% more than the emulsion A.

The reduction in the number of black pixels and simultaneously the increase in the lightness, on the zone of skin covered with a film formed by application of the emulsion INV1 containing a film-forming system according to the invention, demonstrate that the film-forming system according to the invention indeed makes it possible to prevent the adhesion of the microparticles to the skin and also to facilitate their removal by washing, in particular by rinsing with water.

The emulsion INV2 containing a film-forming system according to the invention comprising, as pregelatinized starch, a pregelatinized and hydroxypropylated waxy corn starch exhibits improved barrier effect results with respect to the emulsion INV1. This is because there is a reduction in the number of black pixels of 22.7% and an increase in the lightness of 82.6%.

### Example 3: Sensory Parameters

This example illustrates the change in various sensory parameters observed for aqueous solutions containing different film-forming systems according to the invention, after 48 hours.

All the formulations were produced in the following way:
1) the various components of the film-forming system are weighed out and mixed,
2) the water is heated up to 40° C.,
3) the mixture of ingredients, added gradually, is dispersed using a turbomixer stirring at 2000 revolutions per minute,
4) stirring is allowed to continue until the emulsion is at ambient temperature.

The whiteness descriptor is defined by the palette of colors in FIG. 1. The product is examined under a lamp and compared with the color palette by a panel of evaluators.

The stringiness is an important descriptor for the stage of pick-up of the product. It is evaluated by depositing between 50 and 100 μl of the product on the thumb, which product is subsequently pinched between the thumb and the forefinger slowly, and then by slowly unsticking the forefinger from the thumb: it is then observed whether a connecting string between the thumb and the forefinger is formed (stringy product) or is not formed (nonstringy product). In the case where a connecting string is formed, the distance between the thumb and the forefinger which results in the breaking of the string is evaluated, and the stringy nature is then graded on a scale from 0 to 10: 0 corresponds to the complete absence of a connecting string, 10 corresponds to a connecting string which persists under a maximum distance between the thumb and the forefinger.

The spreading is evaluated by examining the product after having deposited from 50 to 100 μl of the product on the back of the hand, during its spreading in 10 rotations, under a lamp. The less the resistance to movement between the 5^{th} and the 10^{th} turn on the hand, the greater the spreading.

The tack is a descriptor of the afterfeel stage. It is evaluated by examining the product under a lamp after having deposited from 50 to 100 μl of the product on the back of the hand and then having spread it in 10 rotations. A finger is repeatedly pressed and then released on the surface of the hand where the product has been applied. During this movement, the strength of adhesion between the finger and the skin of the hand is evaluated, and grading is carried out on a scale from 0 to 10: 0 corresponds to the complete absence of adhesion, 10 corresponds to an adhesion such that the finger cannot be unstuck from the skin of the hand.

Finally, the penetrating nature of the product is evaluated by an examination under a lamp of the product two minutes after having carried out 10 rotations of the product on the back of the hand, by carrying out sliding over the skin. A panel of evaluators then evaluates the amount of product residue recovered.

Table 6 demonstrates that all of the formulations tested make it possible to obtain a satisfactory sensory parameterization. In particular, a product which is sparingly stringy, with a descriptor of less than or equal to 3, which spreads well, with a spreading descriptor of greater than or equal to 5 and usually of greater than or equal to 7, which is sparingly tacky, with a corresponding descriptor of less than or equal to 4, and which is penetrating, with a corresponding sensory descriptor of greater than or equal to 6, is always obtained. With the exception of certain formulations rich in hydroxyethylcellulose, very white formulations, with whiteness descriptors of greater than or equal to 6, are obtained in all of the formulations. The invention thus makes it possible to obtain products which are satisfactory from a sensory point of view.

TABLE 6
sensory results of aqueous solutions containing
film-forming systems according to the invention

| % PREGEFLO | % Xanthan | | Sensory evaluations after 48 h | | | | |
|---|---|---|---|---|---|---|---|
| CH40 | gum | % HEC | White | Stringiness | Spreading | Tack | Penetrating |
| 2.5 | 0.3 | 0.2 | 7 | 2 | 8 | 2 | 8 |
| 4 | 0.6 | 0.2 | 7 | 3 | 7 | 0 | 8 |
| 4 | 0.3 | 0.8 | 6 | 3 | 5 | 3 | 6 |
| 3.25 | 0.45 | 1 | 3 | 2 | 5 | 3 | 6 |
| 4 | 0.3 | 0.2 | 9 | 2 | 7 | 2 | 7 |
| 4.5 | 0.45 | 0.5 | 6 | 3 | 5 | 4 | 8 |
| 2.5 | 0.3 | 0.8 | 8 | 2 | 7 | 0 | 6 |
| 3.25 | 0.45 | 0.5 | 8 | 3 | 7 | 0 | 9 |
| 2.5 | 0.6 | 0.8 | 5 | 2 | 5 | 1 | 7 |
| 2 | 0.45 | 0.5 | 8 | 1 | 7 | 2 | 7 |
| 4 | 0.6 | 0.8 | 4 | 3 | 7 | 2 | 8 |
| 2.5 | 0.6 | 0.2 | 9 | 3 | 9 | 3 | 7 |
| 3.25 | 0.45 | 0 | 9 | 3 | 8 | 3 | 7 |
| 3.25 | 0.7 | 0.5 | 8 | 2 | 8 | 3 | 8 |
| 3.25 | 0.2 | 0.5 | 9 | 2 | 8 | 4 | 8 |

1. A film-forming system, comprising:
at least one pregelatinized starch,
at least one non-starchy polysaccharide chosen from gums of vegetable origin and gums of microbial origin, and
at least one non-starchy polysaccharide chosen from cellulose derivatives.

2. The film-forming system as claimed in claim 1, characterized in that the pregelatinized starch results from corn, potatoes, wheat, rice, peas, oats, lentils, faba beans, broad beans, beans, chickpeas, or combinations thereof, and preferably results from a waxy starch and in particular a waxy corn, potato or rice starch.

3. The film-forming system as claimed in claim 1, characterized in that the pregelatinized starch is a modified starch, preferably an acetylated, hydroxyethylated, hydroxypropylated, catiomzed, anionized or crosslinked starch.

4. The film-forming system as claimed in claim 1, characterized in that the pregelatinized starch is an acetylated starch.

5. The film-forming system as claimed in claim 1, characterized in that the gums of vegetable origin are gums resulting from plant seeds or exudates, or gums extracted from algae, or derivatives of said gums of vegetable origin.

6. The film-forming system as claimed in claim 1, characterized in that the gums of microbial origin are gums resulting from a microbial fermentation or the derivatives of said gums of microbial origin.

7. The film-forming system as claimed in claim 1, characterized in that the gums of microbial origin are xanthan gums.

8. The film-forming system as claimed in claim 1, characterized in that the cellulose derivatives are modified celluloses, in particular methylcelluloses, hydroxyalkylcelluloses, ethylhydroxyethylcelluloses, methylethylcelluloses, carboxy methylcelluloses, hydroxypropylcelluloses or hydroxypropylmethylcelluloses, carboxy methylcelluloses and hydroxyethylcelluloses being preferred.

9. The film-forming system as claimed in claim 1, characterized in that the cellulose derivatives are hydroxypropylmethylcelluloses.

10. The film-forming system as claimed in claim 1, characterized in that it comprises, and preferably is constituted of:
1 to 12 parts by dry w eight of at least one pregelatinized starch, and
0.01 to 5 parts, preferably 0.01 to 2 parts, by dry w eight of at least one non-starch) polysaccharide chosen from gums of vegetable origin, gums of microbial origin and cellulose derivatives.

11. The film-forming system as claimed in claim 10, characterized in that it comprises:
1 to 12 parts, preferentially of 5 to 11 parts, by dry weight of at least one pregelatinized starch, and
0.01 to 2 parts, preferentially 0.1 to 1 part, by dry w eight of at least one gum of vegetable or microbial origin.

12. The film-forming system as claimed in claim 10, characterized in that it comprises, and preferably is constituted of:
1 to 12 parts, preferentially of 5 to 11 parts, by dry weight of at least one pregelatinized starch,
0.01 to 2 parts, preferentially 0.1 to 1 part, by dry w eight of at least one gum of vegetable or microbial origin,
and 0.01 to 3 parts, preferentially 0.1 to 2 parts, by dry weight of at least one cellulose derivative.

13. The film-forming system as claimed in claim 12, comprising:
6 to 10 parts by dry w eight of at least one pregelatinized starch,
0.2 to 0.8 parts by dry weight of at least one gum of vegetable or microbial origin,
and 1 to 1.5 parts by dry weight of at least one cellulose derivative.

14. (canceled)

15. A process for the manufacture of a film-forming system, comprising the steps of:
a) providing an aqueous solution,
b) heating the aqueous solution to a temperature of between 20° C. and 80° C., preferentially between 20° C. and 50° C. and more preferentially still to a temperature close to ambient temperature, and
c) introducing, with stirring, into the aqueous solution, at least one pregelatinized starch, at least one vegetable gum of vegetable or microbial origin and at least one cellulose derivative, so as to obtain a medium.

16. A cosmetic composition comprising a film-forming system as claimed in claim 1, preferably in a concentration of between 1% and 10% by weight, more preferentially of between 1.5% and 8% by weight and better still between 2% and 6% by weight.

17. A care product for the skin, a makeup product for the eyes, a sun product, a body hygiene product, a hair product or a fragrance, comprising the cosmetic composition according to claim 16.

18. (canceled)

19. A topical cosmetic, comprising the film-forming system according to claim 1.

20. The topical cosmetic according to claim 19, wherein provides for a barrier property on human epidermis.

21. The topical cosmetic according to claim 20, wherein the barrier property protects said human epidermis from atmospheric pollutants.
